(19) United States
(12) Patent Application Publication
(10) Pub. No.: US 2013/0192610 A1
(43) Pub. Date: Aug. 1, 2013

(54) SEALING APPARATUS FOR ANATOMICAL PART IN, E. G., CLOSED CHAMBER THERAPY

(71) Applicants: Emilie Gagnon, St-Basile Le Grand (CA); Christian Lemieux, St-Hubert (CA); Jean-François Jacques, Montreal (CA); Robert McNabb, Rosemere (CA)

(72) Inventors: Emilie Gagnon, St-Basile Le Grand (CA); Christian Lemieux, St-Hubert (CA); Jean-François Jacques, Montreal (CA); Robert McNabb, Rosemere (CA)

(73) Assignees: CENTRE HOSPITALIER DE L'UNIVERSITE DE MONTREAL, Montreal, QC (CA); UNIVERSITE DE MONTREAL, Montreal, QC (CA)

(21) Appl. No.: 13/642,675

(22) PCT Filed: Apr. 20, 2011
(86) PCT No.: PCT/CA2011/050210

§ 371 Date: Mar. 6, 2013

Related U.S. Application Data
(60) Provisional application No. 61/325,929, filed on Apr. 20, 2010.

(51) Int. Cl. A61G 10/00; A61M 37/00

(52) U.S. Cl. CPC A61G 10/00; A61M 37/00
USPC 128/846; 604/23

(57) ABSTRACT

A sealing apparatus for an anatomical part comprises a support collar that comprising an annular body. An elastic sealing membrane is positioned within the support collar. The sealing membrane defines an annular sealed chamber having a given inner diameter in a natural state of the elastic material. A port in the sealing apparatus is in fluid communication with the chamber. The port is adapted to be in fluid communication with a device for reducing pressure in the chamber to increase the given inner diameter of the chamber in a collapsed state, so that an anatomical part may be inserted into the sealing apparatus. A sealing apparatus and accessories are also provided, as is a method for sealing an anatomical part in an inner cavity of an accessory.

### CROSS-REFERENCE TO RELATED APPLICATION(S)

The present application claims priority on U.S. Provisional Application No. 61/325,929, filed on Apr. 20, 2010, and incorporated herein by reference.

### FIELD OF THE APPLICATION

The present application relates to sealing devices and apparatuses of the type used to create a closed chamber between an accessory and an anatomical part, and to a method associated therewith.

### BACKGROUND OF THE ART

It is often desired to enclose an anatomical part in a closed chamber in order to limit the exposure of the anatomical part to environmental conditions. Typically, seals are used to seal off the closed chamber at the entry region of the anatomical part. These seals are typically made of an elastic material, such as rubber, latex, or neoprene, having diameters suited to naturally press against the anatomical part. One of the issues with such seals is the insertion of the anatomical therethrough, which typically involves contact between the anatomical part and the seal or involves a mechanical or manual extension/stretching of the seal to avoid the contact between the anatomical part and the seal.

There are numerous applications requiring closed chambers. For instance, wounds may be subjected to oxygen therapy to accelerate the healing process. In such a case, the anatomical part is enclosed in a closed chamber in which the anatomical part is exposed to air with a high level of oxygen. According to the prior art, rigid casings customized to the anatomical part are typically designed for such applications. Rigid casings usually involve sterilization, disinfection, or like cleaning steps. Accordingly, the cleaning required with rigid casings render this solution non-effective in terms of use and costs.

Prior art seals are not ideal for such applications, as the seals may contact the wounds during the insertion of the anatomical part through the seals.

### SUMMARY OF THE APPLICATION

In accordance with the present application, there is provided a sealing apparatus for an anatomical part, comprising: a support collar; a sealing membrane of an elastic material positioned within the support collar to define an annular sealed chamber therewith having a given inner diameter in a natural state of the elastic material; and means for reducing a pressure in the sealed chamber to increase said given inner diameter of the annular sealed chamber for insertion of the anatomical part in the sealing apparatus.

Therefore, in accordance with a first embodiment of the present application, there is provided a sealing apparatus for an anatomical part, comprising: a support collar comprising an annular body; a sealing membrane of an elastic material positioned within the support collar to define an annular sealed chamber therewith having a given inner diameter in a natural state of the elastic material; and a port in the sealing apparatus in fluid communication with the annular sealed chamber, the port adapted to be in fluid communication with a device for reducing a pressure in the sealed chamber to increase said given inner diameter of the annular sealed chamber in a collapsed state for insertion of the anatomical part in the sealing apparatus.

Further in accordance with the first embodiment, the port comprises a throughbore in a wall in the annular body of the support collar.

Still further in accordance with the first embodiment, the annular body of the support collar defines an inner annular cavity forming part of the annular sealed chamber with the sealing membrane.

Still further in accordance with the first embodiment, the support collar has outer annular channels at opposite ends of the annular body, the outer annular channels being connected to at least one of the sealing membrane and an accessory used with the sealing apparatus.

Still further in accordance with the first embodiment, the apparatus comprises clamping rings releasably connected to the outer annular channels for retaining captive at least one of the sealing membrane and the accessory to the support collar.

Still further in accordance with the first embodiment, wherein the sealing membrane comprises connection rings on opposite ends for being interfaced to the annular body of the support collar.

In accordance with a second embodiment of the present application, the is provided an assembly of a sealing apparatus for an anatomical part and accessories comprising: a pumping device; a sealing apparatus comprising a support collar comprising an annular body, a sealing membrane of an elastic material positioned within the support collar to define an annular sealed chamber therewith having a given inner diameter in a natural state of the elastic material, and a port in the sealing apparatus in fluid communication with the annular sealed chamber, the port being in fluid communication with the pumping device for reducing a pressure in the sealed chamber to increase said given inner diameter of the annular sealed chamber for insertion of the anatomical part in the sealing apparatus; and an accessory connected to the support apparatus to receive the anatomical part passing through the sealing apparatus.

Further in accordance with the second embodiment, the port comprises a throughbore in a wall in the annular body of the support collar.

Still further in accordance with the second embodiment, the annular body of the support collar defines an inner annular cavity forming part of the annular sealed chamber with the sealing membrane.

Still further in accordance with the second embodiment, the support collar has outer annular channels at opposite ends of the annular body, the outer annular channels being connected to at least one of the sealing membrane and an accessory used with the sealing apparatus.

Still further in accordance with the second embodiment, the assembly comprises clamping rings releasably connected to the outer annular channels for retaining captive at least one of the sealing membrane and the accessory to the support collar.

Still further in accordance with the second embodiment, the sealing membrane comprises connection rings on opposite ends for being interfaced to the annular body of the support collar.

Still further in accordance with the second embodiment, the accessory is a bag forming a chamber about the anatomical part.

Still further in accordance with the second embodiment, the bag comprises vent adaptors spaced apart from one another along the bag, and further comprising a gas source connected to the vent adaptors and adapted to create a flow of gas in the bag.

Still further in accordance with the second embodiment, the gas source is an oxygen source, and the flow of gas is a flow of gas with high-oxygen content.

Still further in accordance with the second embodiment, the bag comprises structural components adapted to generally maintain a shape of the bag with a relatively negative pressure in the bag.

Still further in accordance with the second embodiment, the pump device is a bulb connected to the port by tubing.

Still further in accordance with the second embodiment, the pump device comprises a bi-direction valve to reduce and increase a pressure in the annular sealed chamber beyond atmospheric pressure.

In accordance with a third embodiment of the present application, there is provided a method for sealing an anatomical part in an inner cavity of an accessory comprising: receiving a sealing apparatus having an annular body and a sealing membrane inward of the annular body, an opening of the accessory being connected to the sealing apparatus; reducing a pressure in a sealed chamber defined between the annular body and the sealing membrane to increase a diameter of the sealing membrane; inserting the anatomical part in the inner cavity of the accessory by passing through the sealing apparatus; and increasing a pressure in the sealed chamber to press the material of the sealing membrane against the anatomical part at the sealing apparatus; whereby the anatomical part is sealed in the inner cavity of the accessory.

Further in accordance with the third embodiment, increasing the pressure in the sealed chamber comprises increasing the pressure to ambient pressure.

Still further in accordance with the third embodiment, the method comprises further increasing the pressure to a positive pressure relative to an ambient pressure, in the sealed chamber to tighten the sealing membrane against the anatomical part.

Still further in accordance with the third embodiment, reducing and increasing a pressure is performed manually.

Still further in accordance with the third embodiment, the method comprises inducing a flow of gas in the accessory to perform gas exposure therapy on the anatomical part.

### BRIEF DESCRIPTION OF DRAWINGS

FIG. 1 is a perspective view of a sealing apparatus for an anatomical part in accordance with the present disclosure;

FIG. 2 is an assembly view of the sealing apparatus of FIG. 1;

FIG. 3A is a perspective view showing the sealing apparatus in a natural state of a sealing membrane;

FIG. 3B is a perspective view of the sealing apparatus with the sealing membrane in a collapsed state;

FIG. 4A is a perspective view showing the sealing apparatus in the collapsed state of the sealing membrane, with a limb being inserted therein; and

FIG. 4B is a perspective view of the sealing apparatus and limb of FIG. 4A, but with the sealing apparatus in the natural state.

### DESCRIPTION OF PREFERRED EMBODIMENTS

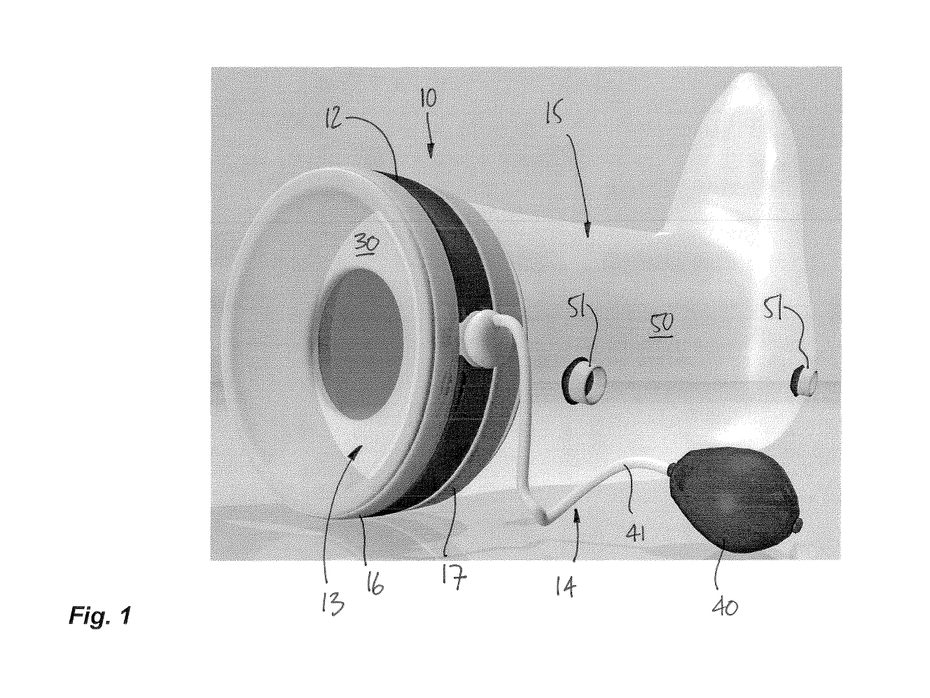

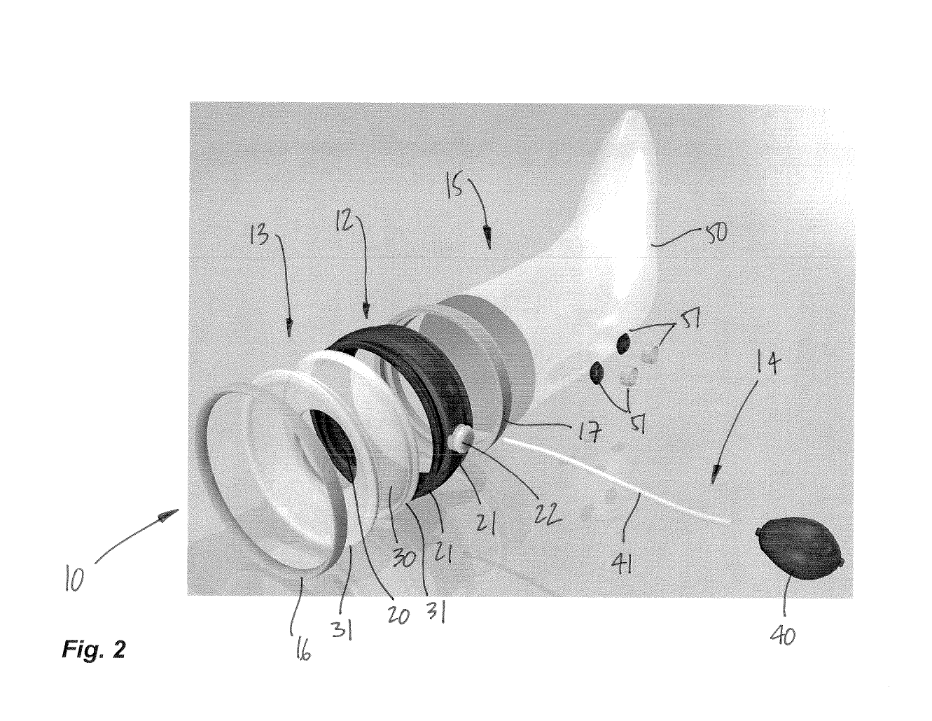

Referring to the drawings, and more particularly to FIGS. 1 and 2, there is illustrated a sealing apparatus 10 for an anatomical part. The sealing apparatus 10 is used in combination with an accessory that typically accommodates an anatomical part, to conceal the anatomical part from the environment. In the illustrated embodiment, the accessory is a boot bag in which the lower leg and foot of a user are subjected to gas exposure therapy, whereby the sealing apparatus 10 seals off the boot bag to keep the a gas in the boot bag. The sealing apparatus 10 may be used in any other appropriate application, such as hyperbaric therapy, hypoxic therapy, etc. In gas exposure, the gas may be oxygen, nitric oxide, nitrous oxide and oxygen mixtures, among numerous other possibilities.

The sealing apparatus 10 has a support collar providing structural integrity to the sealing apparatus 10 and supporting other components thereof. The support collar 12 must be capable of supporting a sealing membrane, and may thus be made of a rigid material or a semi-rigid material. It is also conceived that a non-rigid material can be used for the support collar, or a combination of rigid and non-rigid materials. Moreover, the support collar 12 may consists of hinged segments concurrently forming a ring.

A sealing unit 13 is provided radially inward of the support collar 12. The sealing unit 13 will sealingly contact the anatomical part.

A pump or like pumping device 14 used in assembly with the sealing apparatus 10 adjusts a diameter of the sealing unit 13. An accessory 15 is used in assembly with the sealing apparatus 10 and is connected to one end of the support collar 12 and defines a closed chamber enclosing an anatomical part and sealed by the sealing apparatus 10. As shown in FIG. 2, clamping rings 16 and 17 are provided on opposite ends of the support collar 12, and are used to connect the sealing unit 13 and accessory 15 to the support collar 12, in such a way that the risk of air leakage across the junctions is minimized.

Referring to FIG. 2, the support collar 12 is shown in greater detail. In the illustrated embodiment, the collar 12 has an annular body of circular section, although any other suitable sectional shape may be used as well. An inner annular cavity 20 is provided in an interior of the collar 12. According to an embodiment, the inner annular cavity 20 has a U-shaped section. Outer annular channels 21 are provided on opposite ends of the support collar 12 in the outer surface of the collar 12. A port 22 is positioned on an outer surface of the support collar 12, and is in fluid communication with the inner annular cavity 20. Accordingly, the pump 14 may be connected to the port 22 to control an air pressure in the inner annular cavity 20 once closed off, as will be described hereinafter. It is considered to use any appropriate polymeric material, metallic material and composite for the support collar 12. For instance, polycarbonate is a material well suited for use as the support collar 12.

The sealing unit 13 has an annular sealing membrane 30 positioned between a pair of connection rings 31. The sealing membrane 30 is typically made of an elastomeric material such as silicone, neoprene, chloroprene, polyurethane, natural or synthetic polymers, rubber (e.g., latex rubber) or other rubber-like elastic materials, and has an important level of elasticity. The material of the sealing unit 13 is selected as a function of the desired level of air tightness of the sealing apparatus 10, taking in consideration the application in which the sealing apparatus 10 is used. In an example, the sealing apparatus 10 is used with an accessory 15 forming a pressurized chamber for the anatomical part, whereby an appropriate material must be used for the sealing membrane 30.

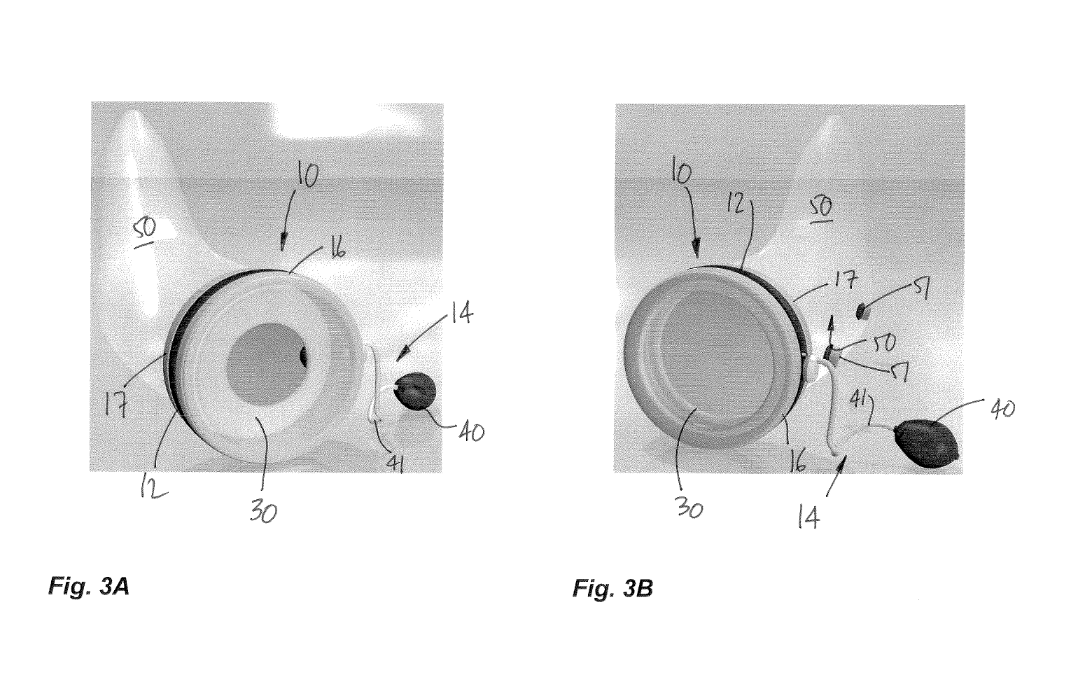

The connection rings 31 are respectively received in the outer annular channels 21 of the support collar 12, with the clamping rings 16 and 17 blocking the rings in the channel 21. Accordingly, a generally airtight chamber (i.e., sealed chamber) is defined between the sealing membrane 30 and the inner surface of the support collar 12. Therefore, a variation of pressure caused by the pump 14 via the port 22 will cause a variation in an inner diameter of the sealing membrane 30. Referring to FIG. 3A, the sealing membrane is shown in its natural state. In this natural state, a pressure inside the sealed chamber of the sealing apparatus 10 is essentially similar to the ambient pressure (e.g., atmospheric pressure). In FIG. 3B, the sealing membrane 30 is in a collapsed state. In the collapsed state, there is a relatively negative pressure in the sealed chamber, causing the sealing membrane 30 to collapse and the inner diameter of the sealing unit 13 to increase.

Therefore, the collapsed state of FIG. 3B is used in order to insert the anatomical part through the sealing apparatus 10, thereby preventing or minimizing contact of the anatomical part with the sealing apparatus 10 during insertion. Once the anatomical part is entered into the sealing apparatus 10 and in a desired position in the accessory 15, the negative pressure effect is released, whereby the sealing membrane 30 regains the natural shape of FIG. 3A, thereby coming into contact with the anatomical part and sealing a junction between the anatomical part and the sealing apparatus 10. It is considered to increase the pressure in the sealed chamber in order to have a pressure greater than the environmental pressure in an expanded state of the sealing membrane 30. This will cause the sealing membrane 30 to press against the anatomical part. However, the pressure is controlled to avoid causing a tourniquet effect on the anatomical part with the sealing membrane 30. The sealing apparatus 10 is for instance selected as a function of the diameter of the sealing membrane 30 in its natural state, with this diameter being smaller than the sectional size of the anatomical part that will be threaded through the sealing apparatus 10. Accordingly, the sealing membrane will naturally contact the anatomical part in its natural state, without being inflated above ambient pressure.

Referring to FIGS. 1 and 2, the pump 14 may be a bulb 40 with a tube 41, and an air valve (not shown). The tube 41 is connected to the port 22 of the support collar 12, and is thus in fluid communication with the sealed chamber. A manual pressure on the bulb 40 will create a suction effect through the tube 41, thereby removing air from the sealed chamber and causing a change of state of the sealing membrane 30 in the manner shown from FIGS. 3A to 3B. In the event that the sealing membrane 30 must also be inflated to the expanded state, the pump 14 has a bidirectional air valve (not shown) used to decrease or increase the pressure in the sealed chamber by the manual actuation of the bulb 40. If other devices are used as an alternative to the manual pump (electric pump, etc.), these other devices may be equipped to operate a suction mode and a pressurizing mode.

The pump 14 is a manual pump in the illustrated embodiment. The air valve typically has a set screw to hold or released the pressure in the sealed chamber of the sealing apparatus 10. It is also considered to use electric pumps or the like with appropriate manometers and automatic (pressure) control system in order to maintain a given pressure in the sealed chamber of the sealing apparatus 10. Typically, it may be desired to maintain given pressures when the sealing unit 13 is in the collapsed or expanded state.

In the illustrated embodiment, the accessory 15 is a boot bag 50 shaped so as to accommodate a leg and foot. Vent adaptors 51 are provided so as to create a flow of oxygen within the bag 50, by appropriately connecting the vent adaptors 51 to an inlet and outlet of a gas source (e.g., oxygen). Accordingly, gas exposure therapy may be performed within the bag 50. It is pointed out that the vent adaptors 51 may also be provided on the sealing apparatus 10. In the illustrated embodiment, the positioning of the vent adaptors 51 on opposite ends of the bag 50 will cause of controlled flow of oxygen in the bag 50 (e.g., 10 L/min). Vent adaptors 51 may likewise be positioned at any other appropriate location on the bag 50. Valves may be provided with the vent adaptors 51 to control the pressure in the bag 50. Therefore, the operation of the sealing apparatus 10 allows a positive pressure to be maintained in the bag 50 during oxygen therapy. Accordingly, the sealing apparatus 10 is used to maintain a pressure differential between the closed chamber defined by the accessory 15 (e.g., bag 50) and ambient pressure. In the case where a negative pressure must be held in the accessory 15, the accessory 15 may be equipped with structural members to preserve its shape.

When used to support a boot for topical oxygen therapy, the sealing apparatus 10 is typically used in combination with leg supports, to lift the leg adjacent to the sealing apparatus 10. In this manner, the leg is concentrically positioned in the support collar 12, and will not thus press on the sealing apparatus 10 because of gravity.

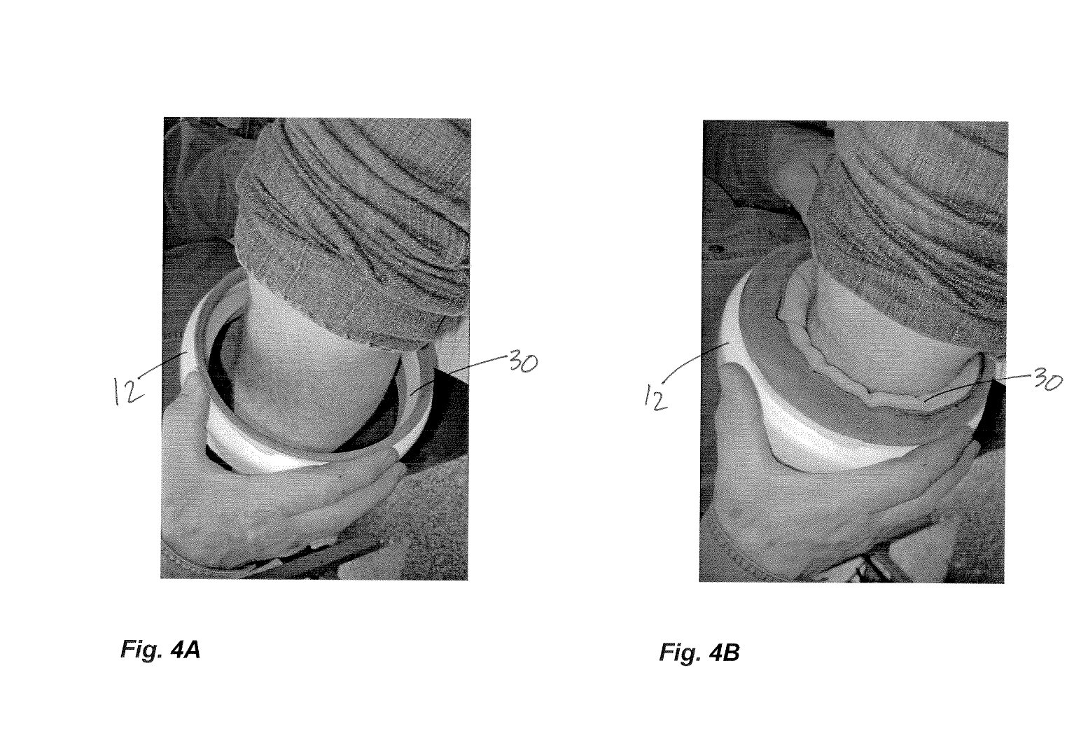

In the embodiment of FIGS. 1-4B, the accessory 15 is a single-use bag. On the other hand, the sealing apparatus 10 may be reused, or the sealing membrane 30 may be replaced for each user.

Although the sealing apparatus 10 is shown in FIGS. 1-4B as being used with a leg, any other anatomical part, such as an arm, a torso, etc., may be used with the sealing apparatus for medical and non-medical applications. For instance, it is considered to use a body suit as the accessory 15, with the sealing apparatus 10 sealing the sealed chamber at the neck. It is also considered to use a pair of the sealing apparatus 10 in combination with a tube of plastic serving as accessory 15, for instance in an oxygen therapy for a knee, or an elbow, among other possibilities.

When selecting a size for the sealing apparatus 10, it is considered to size the inner diameter of the sealing membrane 30 in the normal state as a function of a diameter of the anatomical part against which the sealing membrane 30 will press. More specifically, by selecting the inner diameter of the sealing unit 13 in its natural state to be slightly smaller than the diameter of the anatomical part, the risk of pleats, or non-uniform contact between the anatomical part and the sealing membrane 30 is reduced.

Although the sealing apparatus 10 is shown having a pair of clamping rings 16 and 17, any other appropriate means and method may be used to connect the sealing unit 13 to the support collar 12, such as adhesives, tape, elastics, or the like. Similarly, any appropriate connection device can be used to connect the accessory 15 to the support collar 12.

Now that the various components of the sealing apparatus 10 has been described, a method for sealing an anatomical part in the inner cavity of the accessory 15 is set forth.

A pressure in the sealed chamber defined between the support collar 12 and the sealing membrane is reduced, to increase a diameter of the sealing membrane 13. When the diameter is increased, the anatomical part may be inserted in the inner cavity of the accessory 15 by passing through the support collar 12. Once the anatomical part is suitably inserted, a pressure is increased in the sealed chamber between the support collar 12 and the sealing membrane 13 to press the material of the sealing membrane 30 against the anatomical part, whereby the anatomical part is sealed in the inner cavity of the accessory 15.

The gas exposure therapy may then performed in the accessory 15, by exposing the anatomical part to a gas, wherein in hyperbaric or hypobaric conditions. Moreover, the gas exposure therapy may be combined with skin-treatment therapy, for instance by the application of a medicament (e.g., antibiotics) on the skin prior to the installation of the apparatus 10 and accessory 15.

The inner annular cavity 20 of the support collar 12 is designed to uniformly distribute a collapsing pressure on the sealing membrane 30. This ensures the uniform collapse of the sealing membrane 30, as is shown in FIG. 3B.

The seal performed by the sealing membrane 30 on the anatomical part can be impermeable or semi-permeable to gases (e.g., oxygen or mixtures thereof), and fluids (e.g., water or mixtures thereof).

The sealing apparatus 10 can be used for various applications that necessitate pressure resisting seals. For instance, applications such as pressure chambers for isolating biological or radioactive specimens are considered. The sealing apparatus 10 may also be used for the necks of users whose torsos are confined in so-called “iron lung” respirators.

The sealing apparatus 10 can be used in several application that necessitate fluid resisting seals among which may be briefly mentioned wrists, ankles, and necks for dry suits to protects the human body with the exception of the head, hands and possibly the feet, typically for immersion in water where discomfort would be experienced by a wet suit user. The sealing apparatus 10 may be integrated to helmets, boots and gloves for personal protection when working in and around hazardous liquids. The sealing apparatus 10 may be used with accessories in aeronautical and aerospace applications.

1. A sealing apparatus for an anatomical part, comprising:
a support collar comprising an annular body;
a sealing membrane of an elastic material positioned within the support collar to define an annular sealed chamber therewith having a given inner diameter in a natural state of the elastic material; and
a port in the sealing apparatus in fluid communication with the annular sealed chamber, the port adapted to be in fluid communication with a device for reducing a pressure in the sealed chamber to increase said given inner diameter of the annular sealed chamber in a collapsed state for insertion of the anatomical part in the sealing apparatus.

2. The sealing apparatus according to claim 1, wherein the port comprises a throughbore in a wall in the annular body of the support collar.

3. The sealing apparatus according to claim 1, wherein the annular body of the support collar defines an inner annular cavity forming part of the annular sealed chamber with the sealing membrane.

4. The sealing apparatus according to claim 1, wherein the support collar has outer annular channels at opposite ends of the annular body, the outer annular channels being connected to at least one of the sealing membrane and an accessory used with the sealing apparatus.

5. The sealing apparatus according to claim 4, further comprising clamping rings releasably connected to the outer annular channels for retaining captive at least one of the sealing membrane and the accessory to the support collar.

6. The sealing apparatus according to claim 1, wherein the sealing membrane comprises connection rings on opposite ends for being interfaced to the annular body of the support collar.

7. An assembly of a sealing apparatus for an anatomical part and accessories comprising:
a pumping device;
a sealing apparatus comprising:
a support collar comprising an annular body,
a sealing membrane of an elastic material positioned within the support collar to define an annular sealed chamber therewith having a given inner diameter in a natural state of the elastic material, and
a port in the sealing apparatus in fluid communication with the annular sealed chamber, the port being in fluid communication with the pumping device for reducing a pressure in the sealed chamber to increase said given inner diameter of the annular sealed chamber for insertion of the anatomical part in the sealing apparatus; and
an accessory connected to the support apparatus to receive the anatomical part passing through the sealing apparatus.

8. The assembly according to claim 7, wherein the port comprises a throughbore in a wall in the annular body of the support collar.

9. The assembly according to claim 7, wherein the annular body of the support collar defines an inner annular cavity forming part of the annular sealed chamber with the sealing membrane.

10. The assembly according to claim 7, wherein the support collar has outer annular channels at opposite ends of the annular body, the outer annular channels being connected to at least one of the sealing membrane and an accessory used with the sealing apparatus.

11. The assembly according to claim 10, further comprising clamping rings releasably connected to the outer annular channels for retaining captive at least one of the sealing membrane and the accessory to the support collar.

12. The assembly according to claim 7, wherein the sealing membrane comprises connection rings on opposite ends for being interfaced to the annular body of the support collar.

13. The assembly according to claim 7, wherein the accessory is a bag forming a chamber about the anatomical part.

14. The assembly according to claim 13, wherein the bag comprises vent adaptors spaced apart from one another along the bag, and further comprising a gas source connected to the vent adaptors and adapted to create a flow of gas in the bag.

15. The assembly according to claim 14, wherein the gas source is an oxygen source, and the flow of gas is a flow of gas with high-oxygen content.

16. The assembly according to claim 13, wherein the bag comprises structural components adapted to generally maintain a shape of the bag with a relatively negative pressure in the bag.

17. The assembly according to claim 7, wherein the pump device is a bulb connected to the port by tubing.

18. The assembly according to claim 17, wherein the pump device comprises a bi-direction valve to reduce and increase a pressure in the annular sealed chamber beyond atmospheric pressure.

19. A method for sealing an anatomical part in an inner cavity of an accessory comprising:
receiving a sealing apparatus having an annular body and a sealing membrane inward of the annular body, an opening of the accessory being connected to the sealing apparatus;
reducing a pressure in a sealed chamber defined between the annular body and the sealing membrane to increase a diameter of the sealing membrane;
inserting the anatomical part in the inner cavity of the accessory by passing through the sealing apparatus; and
increasing a pressure in the sealed chamber to press the material of the sealing membrane against the anatomical part at the sealing apparatus;
whereby the anatomical part is sealed in the inner cavity of the accessory.

20. The method according to claim 19, wherein increasing the pressure in the sealed chamber comprises increasing the pressure to ambient pressure.

21.-23. (canceled)
